(19) United States
(12) Patent Application Publication
(10) Pub. No.: US 2018/0154181 A1
(43) Pub. Date: Jun. 7, 2018

(54) MAGNETIC IMAGING GUIDED TREATMENT

(71) Applicant: Aspect Imaging Ltd., Shoham (IL)

(72) Inventor: Uri RAPOPORT, Moshav Ben Shemen (IL)

(73) Assignee: Aspect Imaging Ltd., Shoham (IL)

(21) Appl. No.: 15/736,324

(22) PCT Filed: Jun. 19, 2016
(86) PCT No.: PCT/IL2016/050650

§ 371 Date: Dec. 14, 2017

Related U.S. Application Data
(60) Provisional application No. 62/181,206, filed on Jun. 18, 2015.

(51) Int. Cl. A61N 5/10; G01R 33/48; G01R 33/30; A61B 5/055; A61D 3/00

(52) U.S. Cl. CPC A61N 5/1049; G01R 33/4808; G01R 33/307; A61N 2005/1055; A61D 3/00; A61B 2503/40; A61B 5/0555

(57) ABSTRACT

Devices and method for magnetic resonance guided rotation of an object positioned within a bore of a magnetic resonance device are provided. The devices and method involve obtaining magnetic resonance measurements and rotating the object based on at least one magnetic resonance measurement. Systems comprise at least one irradiation unit configured to deliver radiation to a specified target in a treated object, and a magnetic resonance device (MRD) configured to derive magnetic resonance images of the treated object, the MRD comprising a positioning unit configured to position the treated object at at least two positions, wherein the MRD is configured to detect at least two locations of the specified target at the corresponding at least two positions of the treated object, wherein the system is further configured to adjust the at least one irradiation unit to deliver radiation to the detected at least two locations.

### CROSS REFERENCE TO RELATED APPLICATIONS

This application claims the benefit of and priority to U.S. Provisional Patent Application No. 62/181,206, the entire contents of which are owned by the assignee of the instant application and which are incorporated herein by reference in their entirety.

### FIELD OF THE INVENTION

The invention generally relates to positioning objects for magnetic imaging/radiation. More specifically, the invention relates to a magnetic resonance device (MRD) that allows for three dimensional axial rotation and/or translation of an object during magnetic imaging and/or radiation therapy.

### BACKGROUND OF THE INVENTION

Magnetic resonance devices (MRD) include devices that can perform magnetic resonance measurements, for example, magnetic resonance imaging (MRI) (a.k.a. nuclear magnetic resonance imaging (NMRI)). MRI can use high frequency waves and/or magnetic fields to create images (e.g., obtains three-dimensional sections and/or layered images) of objects (e.g., body organs, tissue for medical diagnosis and research, laboratory research animals). MRI is primarily a noninvasive medical imaging technique used in radiology. MRI can be used to visualize/locate detailed internal structure and systems of a subject (e.g., internal organs of a laboratory animal).

During procedures (e.g., radiation therapy to break kidney stones), MRI can assist in visualizing/locating a precise location on an object/subject to direct radiation. Typically, during MRI radiation, when the precise location is identified, radiation beams are directed to the precise location by way of beam forming and/or rotation of the radiating element about the object. Both of these techniques can be limiting and cause the radiation beam to impinge on a location other than the precise location. For example, a beam emitted by an x-ray source may only be capable of entering an MRD at its opening, thus, limiting the area of the object that the beam can directly impinge upon.

Intensity-modulated radiation therapy (IMRT) is an advanced mode of high-precision radiotherapy that can use computer-controlled linear accelerators to deliver precise radiation doses to a malignant tumor and/or specific areas within the tumor. MRI images can be used to obtain a detailed picture of tumors, tumor borders and/or to specify a three dimensional shape of a tumor and its surrounding vital tissues. Tumor shape, size type and/or location can help a physician to determine how to direct an IMRT beam to impinge substantially upon an ideal location of a tumor and, for example, to avoid healthy tissue.

During IMRT, a medical linear accelerator, which usually includes a gantry arm that holds a treatment head in a desired location and orientation, can deliver an x-ray beam to the tumor from plurality of directions. The accelerator can includes collimator device (e.g., a device for producing a parallel beam of rays or radiation). A computer can adjust and/or modulate an intensity, direction and/or shape of a radiation beam of the collimator device. The adjustments can allows higher doses of radiation to be delivered to the tumor, while sparing the healthy tissue around it.

In many cases the radiation delivery is conducted by a rotation of the treatment head around the radiated subject, either by a plurality of static steps, where the treatment head revolves stops and radiates, where the beam is adjusted according to the specific radiating direction. In other cases the tumor is being radiated, while the treatment head is revolving around the subject and the radiating beam is simultaneously adjusted to according to the treatment head path. In each of these cases, the ability to impinge the radiation beam directly on a desired location of the tumor can be limited, due to, for example, mechanical components of the system blocking a path for the radiation beam to take to the desired location.

MRI imaging can also be used to obtain oblique images. Such oblique MRI slices can be useful in evaluating certain anatomic structures, organs and lesions with oblique topographic orientation (e.g., the heart or the kidney). Some MRI devices can include an orthogonal arrangement of three gradient coils. Typically, MRI devices that include three orthogonal gradient coils are capable of obtaining images in standard axial, coronal and/or sagittal orientation. Images of oblique planes that are rotated around one of the three standard orthogonal axes can be generated by, for example, substantially simultaneous application of two orthogonal gradient fields during three phases (e.g., slice selection, phase encoding and/or signal read-out) of imaging. The obtained images can be as a result of complex computational manipulations and may not provide substantial accuracy due to, for example, an inability to reach the desired position on the subject.

MRI imaging can also be used to obtain to demonstrate effects of gravitational forces on a body system of a subject. Gravitational forces can significantly affect venous return, cardiac output and/or arterial and venous pressures. As a subject shifts from one position to another, for example from lying down to standing up, gravity forces can act on the vascular volume so that blood can accumulate in the lower extremities. The shift in blood volume can decrease thoracic venous blood volume and therefore central venous pressure decreases. This can decrease right ventricular filling pressure (e.g., preload) and lead to, for example, a decline in stroke volume by the Frank-Starling mechanism. Left ventricular stroke volume can also falls because of reduced pulmonary venous return (decreased left ventricular preload). This can causes cardiac output and arterial blood pressure to fall. Obtaining images that demonstrate the effects of the gravitational force can provide important information about the functioning or malfunctions of the various body systems. Typically, MRI images that demonstrate the effects of the gravitational forces are difficult to obtain because, for example, the demonstrated subjects are usually laid, or even restrained, in a single static position, without the option or means to change the subject's orientation in order to employ different directions of gravity forces.

### SUMMARY OF THE INVENTION

The following is a simplified summary providing an initial understanding of the invention. The summary does not necessarily identify key elements nor limit the scope of the invention, but merely serves as an introduction to the following description.

One aspect of the present invention provides systems which comprise at least one irradiation unit configured to deliver radiation to a specified target in a treated object, and a magnetic resonance device (MRD) configured to derive magnetic resonance images of the treated object, the MRD comprising a positioning unit configured to position the treated object at at least two positions, wherein the MRD is configured to detect at least two locations of the specified target at the corresponding at least two positions of the treated object, wherein the system is further configured to adjust the at least one irradiation unit to deliver radiation to the detected at least two locations.

These, additional, and/or other aspects and/or advantages of the present invention are set forth in the detailed description which follows; possibly inferable from the detailed description; and/or learnable by practice of the present invention.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features and advantages thereof, may be understood by reference to the following detailed description when read with the accompanying drawings in which:

FIG. 1 is a high level schematic diagram of an MRD, according to illustrative embodiments of the invention.

FIGS. 2A-2C are high level schematic diagrams of rotation devices for rotating an object within a bore of an MRD, according to illustrative embodiments of the invention.

FIG. 3 is a high level schematic example of an MRD and a positioning system, according to an illustrative embodiment of the invention.

FIGS. 4A-4F are high level schematic diagrams showing examples of components and restraining elements for accommodating an object, according to illustrative embodiments of the invention.

FIG. 5 is a high level schematic illustration of a radiation treatment and imaging system, according to some embodiments of the invention.

FIG. 6 is a high level flowchart illustrating a method 80, according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

In the following description, various aspects of the present invention are described. For purposes of explanation, specific configurations and details are set forth in order to provide a thorough understanding of the present invention. However, it will also be apparent to one skilled in the art that the present invention may be practiced without the specific details presented herein. Furthermore, well known features may have been omitted or simplified in order not to obscure the present invention. With specific reference to the drawings, it is stressed that the particulars shown are by way of example and for purposes of illustrative discussion of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

Before at least one embodiment of the invention is explained in detail, it is to be understood that the invention is not limited in its application to the details of construction and the arrangement of the components set forth in the following description or illustrated in the drawings. The invention is applicable to other embodiments that may be practiced or carried out in various ways as well as to combinations of the disclosed embodiments. Also, it is to be understood that the phraseology and terminology employed herein is for the purpose of description and should not be regarded as limiting.

Devices and method for magnetic resonance guided rotation of an object positioned within a bore of a magnetic resonance device are provided. The devices and method involve obtaining magnetic resonance measurements and rotating the object based on at least one magnetic resonance measurement. Systems comprise at least one irradiation unit configured to deliver radiation to a specified target in a treated object, and a magnetic resonance device (MRD) configured to derive magnetic resonance images of the treated object, the MRD comprising a positioning unit configured to position the treated object at at least two positions, wherein the MRD is configured to detect at least two locations of the specified target at the corresponding at least two positions of the treated object, wherein the system is further configured to adjust the at least one irradiation unit to deliver radiation to the detected at least two locations.

Advantageously, disclosed systems and methods rotate an object to direct magnetization and/or radiation to any precise location of an object, improve accuracy of oblique plane images and obtain information regarding the effects of gravitational forces during radiation.

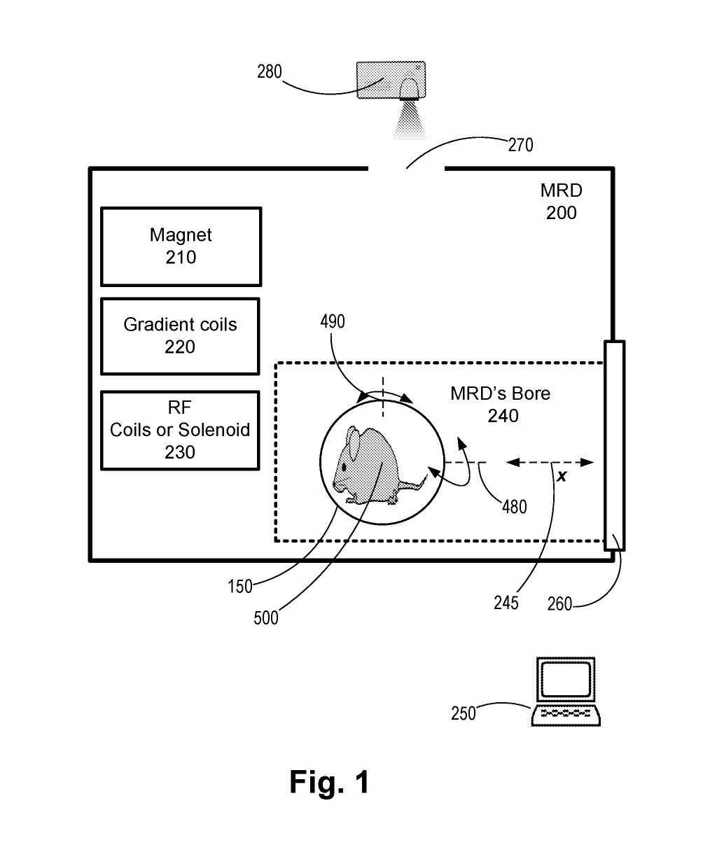

FIG. 1 is a diagram of an MRD 200, according to illustrative embodiments of the invention. The MRD 200 can include a main magnet 210, one or more gradient coils, generally, gradient coils 220, one or more RF coils, generally RF coils 230, a door 260, A radiation opening 270 and a bore 240.

The MRD 200 can be in communication with a controller 250. The radiation device 280 can be positioned relative to the radiation opening 270, such that when emitting radiation beams, the beams can impinge upon an object within the bore 240. In various embodiments, the radiation device 280 is an ultrasound device, an x-ray device, a device that emits a proton beam, a device that emits a gamma ray, or any combination thereof. In various embodiments, the controller 250 includes a joystick, a piezo-electric modulator, a computer mouse, computer keyboard, touchpad, touchscreen and any combination thereof.

The main magnet 210, the gradient coils 220 and the RF coils 20 can be positioned within the MRD 200. The gradient coils 220 can be positioned relative to the main magnet 210 such that magnetic energy that emits from the gradient coils 220 can affect magnetic energy that emits from the main magnet 210.

In various embodiments, the main magnet 210 is a permanent magnet, superconductor magnet and/or other magnet types are known in the art. In some embodiments, the RF coils 230 are solenoids.

The bore 240 can be configured such that it is capable of receiving a rotatable compartment 550. In some embodiments, the bore 240 is a cylinder shape and the compartment 550 is a substantially spherical shape. Any shape of bore 240 may be used in the disclosed systems and methods.

In various embodiments, the MRD 200 includes more than one opening, such that more than one radiating device can emit radiation to the object 505.

In various embodiments, the MRD 200 includes one or more gas inlets into the bore 240. For example, in a laboratory setting where the object 505 is a small animal, a sedation gas can be provided to the small animal via the one or more gas inlets. The gas inlets can be gas inlets as are known in the art.

In various embodiments, the MRD 200 is an MRI device, a Nuclear Magnetic Resonance (NMR) spectroscope, an Electron Spin Resonance (ESR) spectroscope, a Nuclear Quadruple Resonance (NQR) spectroscope functional magnetic resonance imaging (fMRI) or any combination thereof.

It is understood by one of ordinary skill in the art that MRD 200 and its components are described herein for example purposes only and that other configurations of MRD 200 elements, as are known in the art, can be used. For example, the MRD 200 can have any number of gradient coils, RF coils and/or main magnets. The MRD 200 can include other elements, for example, pole pieces, fastening elements and other elements as are known in the art. The bore 240 can include various shapes as are known in the art, for example, shapes that can be dependent upon a type of MRD 200, an expected object type (e.g., lab animal or neonate) and other factors that are known in the art.

In some embodiments, the door 260 may be configured to provide RF shielding of the MRD device and enable insertion of tubes, such as associated with medical devices, into the MRD bore, optionally while maintaining RF shielding.

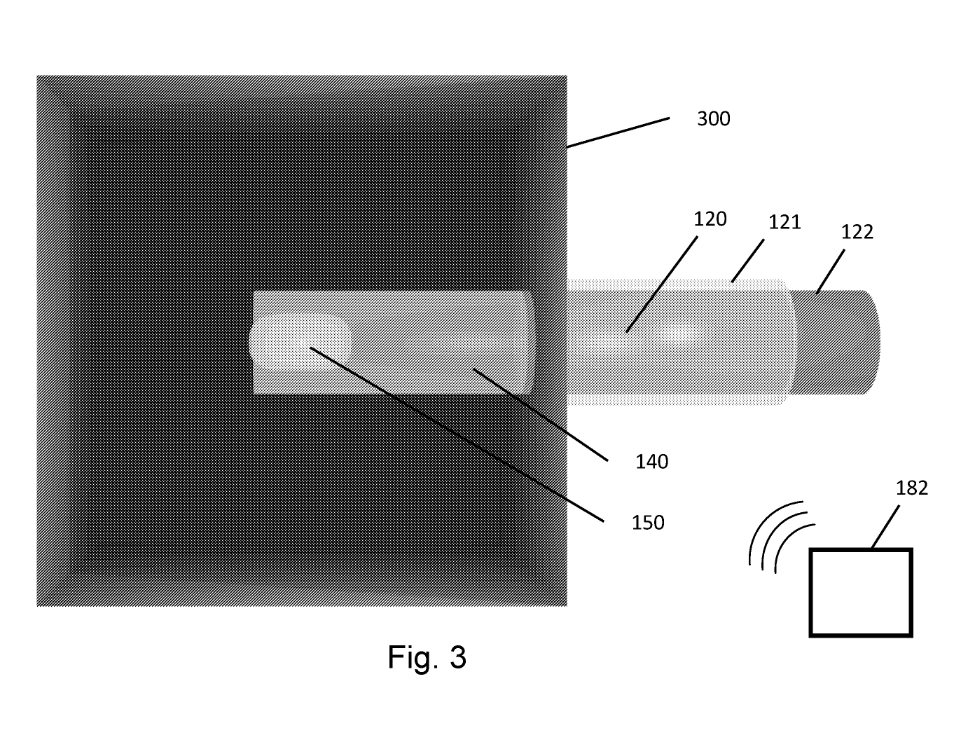

During operation, an object 505 to be imaged/radiated, e.g., a mouse, is positioned within a rotatable compartment 550. The rotatable compartment 550 can be positioned within the bore 240 (e.g., via positioning system 100 as described in further detail below with respect to FIG. 3). With the object 505 positioned within the bore, the MRD 200 can take magnetic resonance measurements the object 505, the radiating device 280 can emit radiation energy to the object 505 and the rotatable compartment 550 can rotate the object 505 about at least two different axes, e.g., a first axis 480 and a second axis 490, while positioned within the bore of the MRD 200.

In some embodiments, the object 505 is rotated about the first axis 490. The first axis 490 can be different from an axis of the bore 480 that permits translation of the rotatable compartment 550 along a length of the bore 240. For example, as shown in FIG. 1, the bore 240 can be a substantially square shape having a length along an axis defined by the x axis 245. In FIG. 1, the x axis 245 is coincident with the second axis 480 such that the second axis 480 is along a longitudinal axis of the bore. As depicted in FIG. 1, the first axis 490 is perpendicular to the second axis 480. In some embodiments, the direction of the first axis 490 is different from the direction of the bore's longitudinal axis.

In some embodiments, the rotatable compartment 550 rotates about axes that allow for a three dimensional and three hundred and sixty degree rotation of the object 505. It is apparent to one of ordinary skill in the art that the first axis 480 and the second axis 490 are examples of axes that the rotatable compartment 550 can rotate about and that the object can rotate about other axes that allow for a three dimensional and three hundred and sixty degree rotation of the object 505. By rotating the object 505, the radiating device 280 can impinge a radiation beam upon any location on the object.

In some embodiments, the controller 250 determines one or more angular distances to rotate the object 505 to place the object 505 in a desired position. For example, it may be desired to rotate the mouse so that its kidney is facing the opening 270, such that, for example, the radiation device 280 can impinge a radiation beam directly on an area of the body that is closets to the kidney. In this example, the controller 250 can determine an angular distance in each respective axis to rotate the mouse such that the kidney is properly positioned. The determination of the angular distances can be made as is known in the art.

In some embodiments, the controller 250 determines the one or more angular distances to rotate the object 505 based on one or more magnetic resonance measurements. For example, when the object 505 is positioned within the bore 240, the MRD 200 can obtain a magnetic resonance measurement of the object 505. The magnetic resonance measurement can be used to determine a region of interest on the object 505 (e.g., a location of a tumor). Once the location of the tumor is identified, that location can be used by the controller 250 to determine the one or more angular distances to rotate the object 505 such that the tumor is facing the opening 270.

In some embodiments, the controller 250 rotates the object in accordance with a predefined protocol.

In some embodiments, a magnetic resonance image (e.g., a first magnetic resonance image) of the object 505 is take, the object is rotated about at least two axes (e.g., the first axis 490 and the second axis 480), and another magnetic resonance image (e.g., a second magnetic resonance image) of the object 505 is taken.

In various embodiments, the object 505 is rotated, magnetically measured, radiated with a beam and/or rotated again. In these various embodiments, order and/or duration of each step can be based on object type, a predefined protocol, and/or a type of procedure to be performed (e.g., MRI guided ultrasound, MRI guided X-ray and other procedures as are known in the art). In these various embodiments, the strength of magnetic field emitted during magnetic measuring and/or the radiation beam can depend upon a procedure type, as is known in the art.

In some embodiments, the object 505 is rotated to provide radiation therapy to the object 505. In some embodiments, the object 505 is rotated to obtain oblique images of the object. In some embodiments, the object 505 is rotated to obtain effects of gravitational force upon a subject's body systems, for example, the gravitational effects on venous return, cardiac output and/or arterial and venous pressures.

In some embodiments, an angle of the radiating beam is modified. In this manner, an additional degree of freedom for radiating an exact location of the object can be obtained.

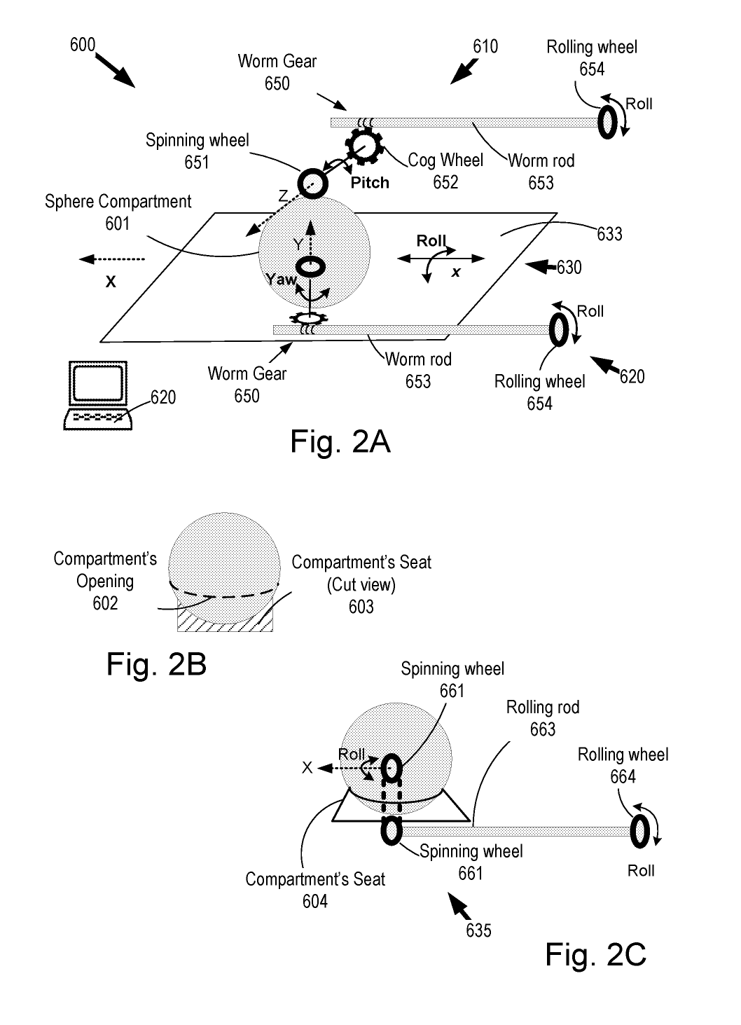

FIGS. 2A-2C are diagrams of rotation devices 600, 670 for rotating an object (e.g., the object 505, as described above with respect to FIG. 1) within a bore (e.g., the bore 240 as described above with respect to FIG. 1) of an MRD (e.g., the MRD 200, as described above with respect to FIG. 1), according to illustrative embodiments of the invention.

FIG. 2A shows an example of a rotation device 600 including a rotatable compartment 601, a compartment seat 604 and rotating elements that can be placed inside of a bore of an MRD (not shown). The rotating elements can include two spinning-wheels 651, 659, two cog wheels 652, 658, two worm gears 650, 655, two worm rods 653,656, two rolling wheels 654, 657 and a pallet 633.

Each rolling wheel 654, 657, can be in contact with its respective worm rod, 653, 656. The worm rods, 653, 656 can be in contact with its respective worm gear 650, 655. Each respective worm gear 650, 655 can be in contact with its respective cog wheel 652, 658. Each respective cog wheel 652, 655 can in contact with its respective spinning wheel 651, 659. Each of the spinning wheels 651 and 659 can be in contact with the rotatable compartment 601.

In various embodiments, additional rotating elements are used. For example, rotating elements can be added to allow rotation of the object about other axes.

With the rotation device 600 placed within a bore of an MRD (not shown), the rotating elements can be actuated from outside of the bore and/or outside of the MRD. For example, the rolling-wheels 654 can extend from the bore and/or MRD to a location that is outside of the MRD via dedicated openings in the bore and/or MRD.

In some embodiments, the rolling-wheels 654 can extend from the bore and/or MRD to a location that is outside of the MRD via a single opening. In some embodiments, the rolling-wheels 654 can extend from the bore and/or MRD to a location that is outside of the MRD via an opening that is also used by a radiation device to provide radiation to the object when the object is positioned within the MRD. In some embodiments, the rolling-wheels 654 can extend from the bore and/or MRD to a location that is outside of the MRD via an entry way of the object into the bore.

In some embodiments, the rolling-wheels 654 can be actuated from an actuator that is positioned within the MRD. In these embodiments, the actuator can include a wired or wireless communication channel with a controller (e.g., controller 250, as described above in FIG. 1) to receive commands that control the actuator and thus, the rotation.

As is apparent to one of ordinary skill in the art, other configurations for location of the rotating elements, number of rotating elements, actuator location and/or number of actuators can be used and are within the scope of the configurations contemplated for rotating an object within an MRD.

In some embodiments, MRD openings that are dedicated to the rotating elements can include magnetic and RF shielding.

In some embodiments, the rotatable compartment 601 is a sphere shape 601. In various embodiments, the rotatable compartment 601 is a cylinder, a hemisphere, an ellipsoid, or a box shape. In some embodiments, the rotatable compartment 601 has any shape that is suitable for accommodating the object and inserting into a bore of a desired MRD type.

As shown in FIG. 2B, in some embodiments, the rotatable compartment 601 has a reversible opening 602 for insertion of an object and/or to open and close the rotatable compartment 601. In some embodiments, the rotatable compartment 601 can be positioned within a seat 603. The seat 603 can be shaped to prevent the rotatable compartment 601 from translating when the rotatable compartment 601 is rotated within the seat 603.

In some embodiments, the seat 603 is coupled to a bore of a MRD. In some embodiments, the seat 603 is coupled to a device (e.g., positioning system 100 as described below with respect to FIG. 3) that inserts the rotatable compartment 601 into a bore of a MRD. In some embodiments, the seat 603 is coupled to the pallet 633. In these embodiments, the pallet 633 can be detachably coupled to a MRD, such that the pallet 633 with the seat 603 can be translated while within a bore of a MRD. As is apparent to one of ordinary skill in the art, the pallet 633/seat 603 configuration for allowing translation of the rotatable compartment 601 within a bore of a MRD is an example only and that other devices as are known in the art can be used to translate the rotatable compartment 601 within a bore of the MRD.

FIG. 2C shows an example of a rotation device 670 including the rotatable compartment 601, a compartment seat 604, a spinning wheel 661, a spinning wheel 662, a rolling rod 663 and a rolling wheel 664.

The rolling wheel 664 can be in contact with the rolling rod 663. The rolling rod 663 can be in contact with the spinning wheel 661. The spinning wheel 661 can be in contact with the spinning wheel 662. The spinning wheel 661 can be in contact with the rotatable compartment 601.

FIGS. 2A and 2C show an example of three orthogonal rotation directions for rotation of the rotatable compartment 601. The three orthogonal rotation directions can allow any orientation for the rotatable compartment 601. The three orthogonal rotation directions can be pitch, yaw and roll.

The roll can be a rotation about the X axis (e.g., a first axis). The roll can be modified by actuating the rolling wheel 664 and/or by moving the pallet 633. The yaw can be a rotation about the Y axis (e.g., a second axis). The Y axis can be orthogonal to the X axis. The yaw can be modified by actuating the rolling wheel 657. The pitch can be a rotation about the Z axis (e.g., a third axis). The Z axis can be orthogonal to both the X axis and the Y axis. The pitch can be modified by actuating the rolling wheel 654.

It is apparent to one of ordinary skill in the art that the axes and rotational directions described above are for illustrative purposes only and that other axes and/or rotational directions can be defined for rotating the object, as is known in the art.

In some embodiments, the X axis is coincident with a longitudinal axis of a bore of an MRD (not shown). In these embodiments, the rolling wheels 654, 657 can extend to a location outside of the MRD along the longitudinal axis of the bore via a single opening of the bore (e.g., through an opening of an open bore MRI device) and actuated by turning rolling wheels 654 and 657. In this manner, the rotatable compartment 601 can be rotated in to a desired orientation by manipulating rotation elements that are substantially in the same location, which can provide ease of use, if for example, the rolling wheels 654 and 657 are manually actuated.

FIG. 3 is an example of an MRD 300 and a positioning system 100, according to an illustrative embodiment of the invention. The MRD 300 can include an opening 125. The positioning system 100 can include an outer shaft 121 and an inner shaft 122. When the positioning system 100 is inserted into the MRD 300, the outer shaft 121 and/or the inner shaft 122 can have a proximal portion 120 and a distal portion 140. The proximal portion 120 can be positioned outside of the MRD 300 and the distal portion 140 can be positioned within the device 300.

The outer shaft 121 can be positioned such that it remains substantially immovable during operation of the MRD 300. The inner shaft 122 can be translatable and/or rotatable within the outer shaft 121. An object (e.g., the object 505 as described above in FIG. 1) and/or rotatable compartment 155 (e.g., an accommodation component) can be coupled to the inner shaft 112, such that translation and/or rotation of the inner shaft 112 causes translation and/or rotation of the object and/or rotatable compartment 155.

The outer shaft 121 can be configured for easy gripping, stabilizing and/or handling of the positioning system 100. The outer shaft 121 and/or the inner shafts 122 can include indicia for indicating and accurately and reproducibly position positioning system 100. In these embodiments, the indicia can be presented in a manner selected from the group consisting of graphically presented on the proximal portion, digitally presented on a display, or any combination thereof.

In various embodiments, translocating the rotatable compartment 150 is done mechanically, electrically, pneumatically and/or hydraulically. In various embodiments, translocating the rotatable compartment 150 is done manually or automatically. In some embodiments, the rotatable compartment 155 is controlled via a piezo-operated joystick. In some embodiments, the rotatable compartment 155 translocates according to a predetermined protocol, translocating it in predetermined parameters, for example, rate of translocation, extent of rotational displacement, liner extent of translocation, or any combination thereof. Predetermined protocols can include various number of iterations, repeating such protocol a number of consecutive times, or alternating between at least two different protocols of

In some embodiments, rotatable compartment 155 is isolated from an environment surrounding rotatable compartment 155 with respect to, for example, a parameter selected from the group consisting of temperature, fluid composition, humidity, light and any combination thereof.

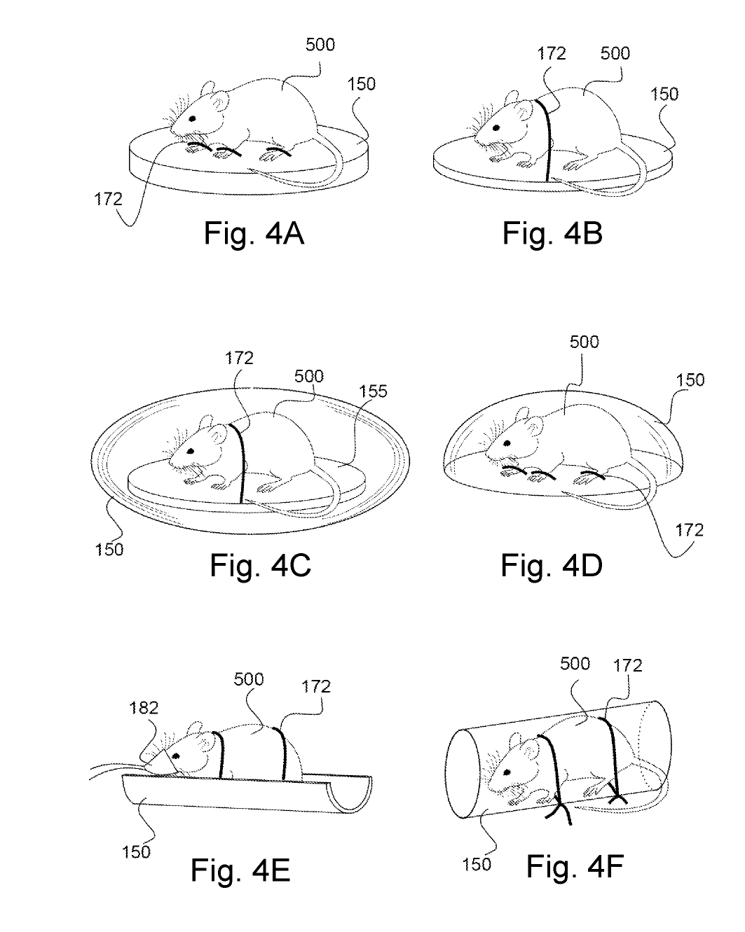

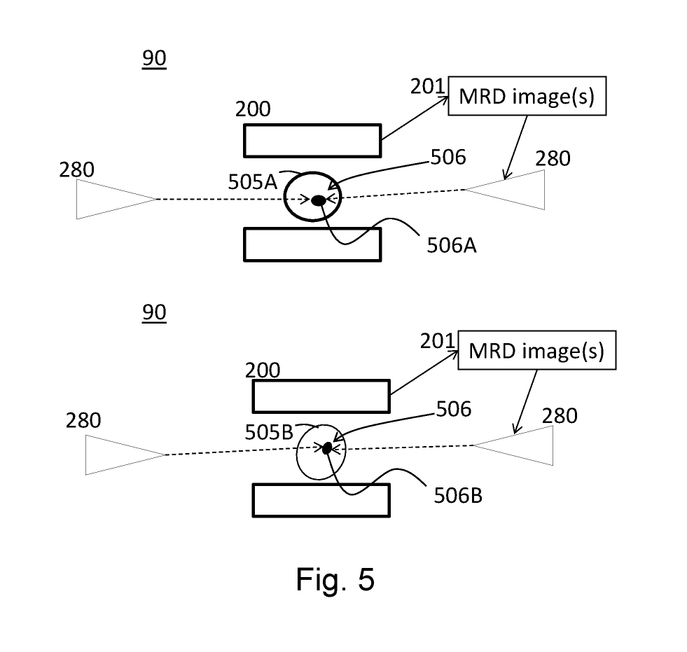

FIGS. 4A-4F are diagrams showing examples of components 150 and restraining elements 172 for accommodating an object, according to illustrative embodiments of the invention. As shown in FIGS. 4A-4F, some of the components 150 can be disposed within a rotatable component (e.g., rotatable compartment 601 as described above in FIG. 1) and some of the components 150 can be inserted directly within a bore of an MRD. FIG. 4A shows a component 150 that is a pallet-like form, having multiple restraining elements 172 that can immobilize an object 500, which is depicted is a mouse. FIG. 5B shows that the component 150 can be various widths and sizes and in some embodiments, only one restraining element 172 is used. In various embodiments, the restraining element 172 is glue, grooves, projections, muzzle, or any combination thereof In some embodiments, the restraining element 172 is any barricade or barrier capable of immobilizing any examined object.

FIGS. 5C and 5D shows the component 150 having a shell-like shape. FIG. 5C shows the component 150 having the form of a sphere with an additional surface disposed therein (e.g., a bed) 155, for stabilizing the object. FIG. 5D shows the component having a hemisphere shape.

FIG. 4E shows the component 150 having a rounded bed-like shape and provided with life support system (LSS) 182. The LSS can be provided for enabling a controlled environment for the object, for example, in the absence of an enclosed compartment for insertion into a bore. In some embodiments, the subject is connected to life support system (LSS), for example: breathing/anesthetic mask (182, as shown in FIG. 5E). According to some embodiments the breathing aid and/or anesthetic fluids are provided to the subject via perforations in the compartment (not shown).

FIG. 4F shows a compartment 150 having shape of an open cylinder. In this embodiment, the restraining elements 172 extend beyond an outer surface of the compartment 550 and are adapted to have a changeable length and tightness level. In various embodiments, once examined object 505 is placed, a closing door (not shown) for the cylinder might be provided, transforming it into a closed compartment.

In some embodiments, the restraining elements 172 length are adjustable.

In some embodiments, the compartment's inner environment is isolated from the environment surrounding the accommodation component with respect to a parameter selected from the group consisting of temperature, fluid composition, humidity, light and any combination thereof.

FIG. 5 is a high level schematic illustration of a radiation treatment and imaging system 90, according to some embodiments of the invention.

Systems 90 may comprise at least one irradiation unit 280 (as described above) configured to deliver radiation and/or particles to a specified target 506 in a treated object 505 (see above), and a magnetic resonance device (MRD) 200 (see above) configured to derive magnetic resonance images 201 (shown schematically, see also above) of treated object 505. MRD 200 comprises a positioning unit 150 (see above, not shown in FIG. 5) which is configured to position treated object 505 at at least two positions 505A, 505B. MRD 200 is configured to detect at least two locations 506A, 506B of specified target 506 at the corresponding at least two positions 505A, 505B of treated object 505. The detection is carried out on MRI images 201 derived by MRD 200, which enable detection of targets 506 in objects 505, e.g. detection of a liver in a mouse (see examples above). System 90 is further configured to adjust at least one irradiation unit 280 to deliver radiation to the detected at least two locations 506A, 506B, e.g., compensating for gravitational effects as described above. The radiation may comprise electromagnetic radiation or protons, as described above, irradiation unit 280 may comprise at least two subunits (illustrated schematically as two triangles in FIG. 5) configured to deliver radiation to specified target 506 from corresponding at least two directions, e.g., directions perpendicular to the MRD bore, directions along the MRD bore, etc. Objects 505 may comprise any of lab animals (mouse, rat, etc.), parts of the human body (e.g., wrist, head, foot etc.) and even humans. Specified target 506 may be e.g., a tumor. MRD 200 may be configured to derive MRI images 201 prior to irradiation, to determine the exact location of target 506A, 506B at any position 505A, 505B, to enable effective radiation delivery at any of multiple postures of objects 505, to take into account shifts of the targets due to gravitational and posture effects. Multiple positions and locations may be used and even continuous or step-wise adjustments of position of the object (and the location of the target) may be imaged and irradiated. Moreover, irradiation unit 280 may be configured to move and direct radiation and/or particles to target 506 according to its imaged position, which may change continuously or stepwise. Irradiation (radiation and/or particles) may be delivered from any angle to object 505 which is available according to the structure of MRD 200, i.e., from any number of available directions. Irradiation unit 280 may comprise internal radiation sources, position close to, upon or in object 505, which may be configured to deliver additional radiation and/or particles, and may be control in some cases according to the position of object 505 and location of target 506.

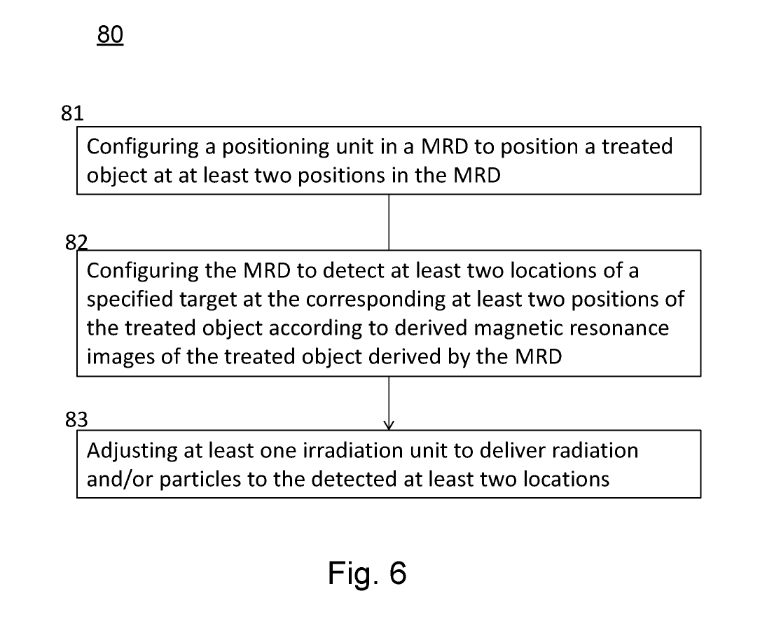

FIG. 6 is a high level flowchart illustrating a method 80, according to some embodiments of the invention. Method 80 may be implemented by system 90 and may comprise any of the method steps presented above. Methods 80 may comprise configuring a positioning unit in a MRD to position a treated object at at least two positions in the MRD (stage 81), configuring the MRD to detect at least two locations of a specified target at the corresponding at least two positions of the treated object according to derived magnetic resonance images of the treated object derived by the MRD (stage 82), and adjusting at least one irradiation unit to deliver radiation and/or particles to the detected at least two locations (stage 83). Systems 90 and methods 80 may be implemented using any of the elements and stages described above and in the figures.

In the above description, an embodiment is an example or implementation of the invention. The various appearances of “one embodiment”, “an embodiment”, “certain embodiments” or “some embodiments” do not necessarily all refer to the same embodiments. Although various features of the invention may be described in the context of a single embodiment, the features may also be provided separately or in any suitable combination. Conversely, although the invention may be described herein in the context of separate embodiments for clarity, the invention may also be implemented in a single embodiment. Certain embodiments of the invention may include features from different embodiments disclosed above, and certain embodiments may incorporate elements from other embodiments disclosed above. The disclosure of elements of the invention in the context of a specific embodiment is not to be taken as limiting their use in the specific embodiment alone. Furthermore, it is to be understood that the invention can be carried out or practiced in various ways and that the invention can be implemented in certain embodiments other than the ones outlined in the description above.

The invention is not limited to those diagrams or to the corresponding descriptions. For example, flow need not move through each illustrated box or state, or in exactly the same order as illustrated and described. Meanings of technical and scientific terms used herein are to be commonly understood as by one of ordinary skill in the art to which the invention belongs, unless otherwise defined. While the invention has been described with respect to a limited number of embodiments, these should not be construed as limitations on the scope of the invention, but rather as exemplifications of some of the preferred embodiments. Other possible variations, modifications, and applications are also within the scope of the invention. Accordingly, the scope of the invention should not be limited by what has thus far been described, but by the appended claims and their legal equivalents.

1. A method for magnetic resonance guided rotation of an object positioned within a bore of a magnetic resonance device (MRD), the method comprising:
obtaining, via the MRD, a first magnetic resonance measurement of the object positioned within the bore of the MRD;
rotating the object about at least two different axes while the object is positioned within the bore of the MRD based on the first magnetic resonance measurement; and
obtaining, via the MRD, a second magnetic resonance measurement.

2. The method of claim 1 wherein the rotating positions the object at a desired location, the desired location based on a type of radiating element that directs radiation upon the object, a location of a radiation device relative to the location of the object, a type of the object, a desired point on the object to be radiated, or any combination thereof.

3. The method of claim 1, further comprising:
radiating, via a radiation device, the object while the object is positioned within the bore of the MRD;
obtaining, via the MRD, a third magnetic resonance measurement of the object positioned within the bore of the MRD; and
rotating the object about at least one of the two different axes such that the radiation emitted by the radiation device impinges upon the object at a predetermined location on the object based on the third magnetic resonance measurement.

4. The method of claim 1, wherein rotating further comprises:
rotating the object about a first axis of the at least two different rotation axes; and
rotating the object about a second axis of the at least two different rotation axes, after the object is rotated about the first axis.

5. The method of claim 1, wherein rotating further comprises:
rotating the object about a first axis of the at least two different rotation axes; and
rotating the object about a second axis of the at least two different rotation axes, wherein rotating about the first axis and rotating about the second axis occurs substantially simultaneously.

6. The method of claim 1, wherein the rotating further comprises controlling the rotation of the object from outside of the bore.

7. The method of claim 1, wherein the method further comprises translating the object along an axis parallel to longitudinal axis of the bore and wherein the translating is at least one of simultaneous to the rotating or sequential to the rotating.

8. The method of claim 1, wherein the at least two different rotation axes are orthogonal to one another.

9. The method of claim 1, wherein the at least two different rotation axes cross one another, at a single point.

10. The method of claim 1, further comprising calibrating the MRD to compensate for an unintentional displacement of the object or the objects internal parts caused by gravitational force.

11. The method of any preceding claim, wherein rotating further comprises positioning the object such that the second magnetic resonance image is an oblique image of the object.

12. A method for obtaining magnetic resonance images of an object positioned within a bore of a magnetic resonance device (MRD), the method comprising:
rotating the object around at least one axis, while the object is positioned within the bore, wherein the at least one rotation axis comprises a direction which is different from direction of longitudinal axis of the bore; and
obtaining a magnetic resonance image.

13. A system for positioning an object within a bore of a magnetic resonance device (MRD), the system comprising:
a rotation device, the rotation device comprising:
a rotatable compartment for accommodating the object, the compartment is configured to receive the object and be inserted into the bore of the MRD and
at least two rotating elements coupled to the compartment, each rotating element of the at least two rotating elements allow for the compartment and object disposed therein to rotate about at least two different axis; and
an MRD to obtain one or more magnetic resonance measurements of the object.

14. The system of claim 13 further comprising a radiation device to radiate the object while the object is positioned within the bore of the MRD.

15. The system of claim 13, wherein at least one of the rotating elements is configured to rotate the compartment about an axis with a direction which is different from direction of a longitudinal axis of the bore, such that the object rotates about the axis.

16. The system of any of claims 13 further comprising a seat configured to be secured to the bore.

17. The system of any of claims 13 further comprising a seat configured to be secured to a longitudinal-element of the MRD inserted into the bore, the longitudinal element allows at least one of:
the seat's translation along longitudinal axis of the bore and
the seat's rotation around longitudinal axis of the bore, controlled by one of the rotating-elements.

18. The system of any of claims 13, further comprising a mechanical actuator coupled to the rotating elements to cause the compartment and object disposed therein to rotate.

19. The system of any of claims 13, wherein at least one of the at least two rotating-elements comprises a worm-gear, a rolling-gear, or two orthogonal cog wheels.

20. (canceled)

21. (canceled)

22. The system of any of claims 13, wherein the object is detachably secured to the compartment with at least one securing element.

23-26. (canceled)
